(19) United States
(12) Patent Application Publication
(10) Pub. No.: US 2021/0050117 A1
(43) Pub. Date: Feb. 18, 2021

(54) OPERATIONS MANAGEMENT BASED ON PREDICTED OPERATIONS EFFECTIVENESS

(71) Applicant: International Business Machines Corporation, Armonk, NY (US)

(72) Inventors: William Ogallo, Nairobi (KE); Aisha Walcott, Nairobi (KE); Charity Wayua, Nairobi (KE); Komminist Weldermariam, Ottawa (CA)

(21) Appl. No.: 16/539,162

(22) Filed: Aug. 13, 2019

(51) Int. Cl. G16H 70/20; G16H 20/40; G16H 50/20

(52) U.S. Cl. CPC G16H 70/20; G16H 50/20; G16H 20/40

(57) ABSTRACT

Methods and systems for monitoring an ongoing procedure are described. In an example, a processor can receive a first set of data indicating a current context of a procedure being performed by a first entity on a second entity. The processor can determine a first score based on the first set of data, where the first score can indicate an effectiveness of the procedure. The processor can identify a set of requirements based on the first score. The set of requirements can be associated with a second score indicating a target effectiveness of the procedure. The processor can generate a recommendation for the first entity to perform the set of requirements to achieve the second score.

### BACKGROUND

The present application relates generally to computers and computer applications, and more particularly to computer-implemented medical systems that can dynamically monitor an ongoing medical procedure to allocate and distribute resources for the ongoing medical procedure and potential post-procedure activities.

Medical procedures being performed by medical professionals can be supported by various technologies such as sensors, surveillances, robots, Internet of Things (IoT) devices, etc. Data collected by these technologies can be used by the medical professionals to support ongoing medical procedures. These data can also be stored and processed to provide reference information for future procedures.

### SUMMARY

In some examples, a method for monitoring an ongoing procedure is generally described. The method can include receiving, by a processor, a first set of data indicating a current context of a procedure being performed by a first entity on a second entity. The method can further include determining, by the processor, a first score based on the first set of data. The first score can indicate an effectiveness of the procedure. The method can further include identifying, by the processor, a set of requirements based on the first score. The set of requirements can be associated with a second score indicating a target effectiveness of the procedure. The method can further include generating, by the processor, a recommendation for the first entity to perform the set of requirements to achieve the second score.

In some examples, a system for monitoring an ongoing procedure is generally described. The system can include a memory configured to store a first set of data indicating a current context of a procedure being performed by a first entity on a second entity. The system can further include a processor comprising hardware, and the processor can be configured to be in communication with the memory. The processor can be configured to determine a first score based on the first set of data. The first score can indicate an effectiveness of the procedure. The processor can further be configured to identify a set of requirements based on the first score. The set of requirements can be associated with a second score indicating a target effectiveness of the procedure. The processor can further be configured to generate a recommendation for the first entity to perform the set of requirements to achieve the second score.

In some examples, a computer program product for monitoring an ongoing procedure is generally described. The computer program product may include a computer readable storage medium having program instructions embodied therewith. The program instructions may be executable by a processing element of a device to cause the device to perform one or more methods described herein.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 is a diagram showing an example computer system that can implement operations management based on predicted operations effectiveness in one embodiment.

FIG. 2 is a diagram showing additional details of the example computer system of FIG. 1 that can implement operations management based on predicted operations effectiveness in one embodiment.

FIG. 3 is a flow diagram illustrating a process relating to operations management based on predicted operations effectiveness in one embodiment.

FIG. 4 illustrates a schematic of an example computer or processing system relating to operations management based on predicted operations effectiveness in one embodiment.

FIG. 5 depicts a cloud computing environment according to an embodiment of the present invention.

FIG. 6 depicts abstraction model layers according to an embodiment of the present invention.

### DETAILED DESCRIPTION

In some examples, a medical procedure being performed by a medical professional (or medical service provider) may require tasks such as coordinating and communicating with entities (e.g., other medical professionals), timely resource allocation and distribution, timely information exchange, etc. The medical professional performing the medical procedure may be given erroneous resources and/or may lack critical supplies if poor coordination between the medical professional and the entities occurs. To be described in more detail below, the system and methods described in accordance with the present disclosure utilizes data collected from various sensors and IoT devices to evaluate a context of an ongoing medical procedure. The evaluation of the context allows a computer device to autonomously determine resources and actions that may provide assistance to the ongoing medical procedure, autonomously respond to spontaneous events during the ongoing medical procedure, and provide recommendations in response to the spontaneous events.

For example, the computer device can evaluate a current context of an ongoing medical procedure by determining an effectiveness score based on data collected from various sensors and IoT devices. The procedure effectiveness score can be presented to the medical professional performing the ongoing medical procedure for various assessment purposes, and a recommendation can be provided by the computer device to improve the procedure effectiveness score. In another example, the data collected by the sensors and IoT devices can be utilized to identify resources (e.g., supplies, tools, care providers, guidelines, etc.) that may be required for the ongoing medical procedure at a current time and also at a future time (e.g., required resources for post-procedure activities).

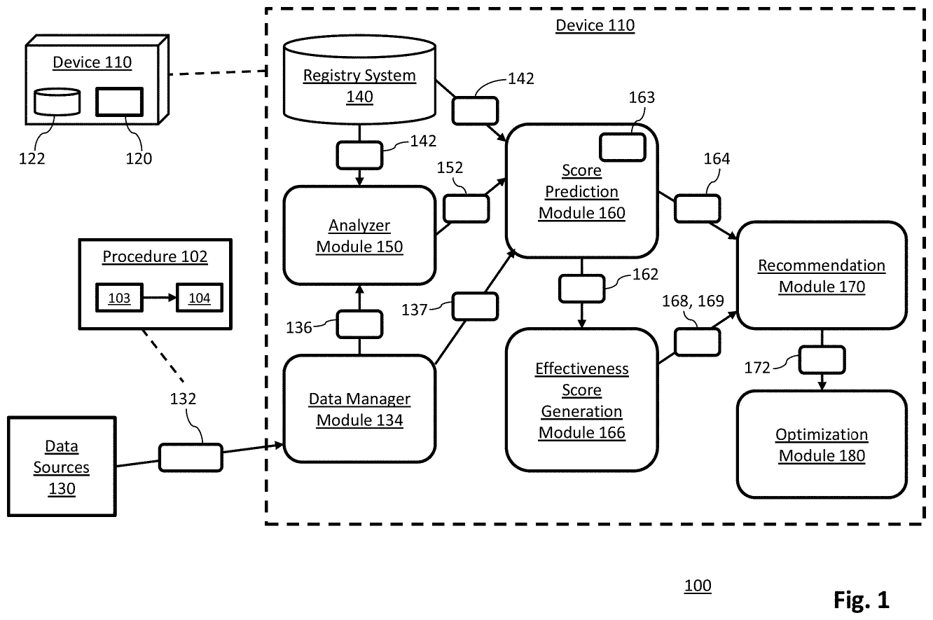

FIG. 1 is a diagram showing an example computer system 100 that can implement operations management based on predicted operations effectiveness in one embodiment. The system 100 can include a computer device (“device”) 110 and one or more data sources 130. The data sources 130 can include, for example, one or more sensors and/or devices such as cameras, microphones, accelerometers, radio frequency identification (RFID) tags, infrared (IR) sensors, etc. The device 110 can be, for example, a server, a desktop or laptop computer, and/or other types of computer devices. The device 110 can include a hardware processor (“processor”) 120 and a memory 122 configured to be in communication with each other. The device 110 can include one or more hardware modules and/or software modules that can be implemented by hardware. For example, the device 110 can include a registry system (or module) 140, an analyzer module 150, a data manager module 134, a score prediction module 160, an effectiveness score generation module 166, a recommendation module 170, and an optimization module 180. The functionality of the modules 140, 150, 134, 160, 166, 170, 180 will be described below. The memory 122 can be configured to store instructions including code that can be executable by the processor 120 to perform the methods being described herein. In an example embodiment, the device 110 can be a component of a cloud computing platform, and data being generated and outputted by the device 110 can be transmitted to another device through a network (e.g., the Internet). For example, the device 110 may be a component of a cloud computing platform and can be configured to perform the methods being described herein to generate an output, and can be configured to transmit the output to a computer device in, for example, a hospital to provide information, assistance, and recommendation to a medical professional performing a medical procedure in the hospital.

The registry system 140 can be configured to stored and maintain profiles for entities including provider entity (e.g., medical professionals) and client entities (e.g., patients), resources (e.g., supplies, tools, guidelines, etc.), and/or procedures (e.g., different types of surgeries, operations, procedures). Client entity profiles can include data indicting physical and cognitive status, preferences, medical history, behavioral signals, self-management capacity, patient-specific needs and goals, and/or other information relating to one or more client entities. Provider entity profiles can include data indicating knowledge, skills, responsibilities, experience, preferences, historical actions and decisions, and/or other information relating to one or more provider entities. Resource profiles can include data indicating availability, equipment age, usage instructions, protocols, guidelines, and/or other information relating to one or more resources. Procedure profiles can include historical and real-time (or current) information such as activities performed, progress, duration, results, movements, communications between provider entities, interactions with resources, and/or other information relating to one or more procedures. The profiles stored in the registry system 140 may include data relating to one another, such that the profiles can be stored as components of various types of databases or models (e.g., relational database, hierarchical network, ontology network, and/or other types of database or networks).

The registry system 140 can also store data from, for example, electronic health record systems, administrative claims systems, and health provider registries. Other types of data that could be stored in the registry system 140 include health systems data such as level of fragmentation, health workforce, supply chain, care protocols, infrastructure, payments, communications logs, health information exchange logs, interpersonal communication, and information transfer. The data being stored in the registry system 140 can be encrypted, and various data security and data protection schemes may be implemented with the registry system 140 to prevent unauthorized access to the data being stored in the registry system 140. Further, permission by data owners, such as medical professional and patients, may be required for storing any data in the registry system 140. Further, users such as entities including medical professionals and patients may choose to participate or not participate (opt-in or opt-out) in the monitoring being performed by the system 100. In one embodiment, the registry system can be based on a blockchain framework implementation (e.g., Hyperledger Fabric) to ensure provenance, trust, traceability, and governance of all data in the registry.

In an example, a procedure 102 can be an ongoing medical procedure being performed by a first entity 103 (e.g., a provider entity such as a medical service provider), and the procedure 102 can be an operation on a second entity 104 (e.g., a client entity such as a patient). The processor 120 can identify, in the registry system 140, a first entity profile of the first entity 103, a second entity profile of the second entity 104. The processor 120 can further identify a procedure profile of the procedure 102, where the procedure profile can include data indicating a type of the procedure 102 and a set of resources that may be needed to perform the procedure 102. The processor 120 can further identify a resource profile of the set of resources indicated by the procedure profile. The processor 120 can retrieve the profiles identified from the registry system 140, and aggregate the identified profiles into profile data 142. The processor 120 can distribute the profile data 142 to the analyzer module 150 and the score prediction module 160. In some examples, the processor 120 can autonomously identify the profiles in the registry system 140 in response to a start of the procedure 102. In some examples, the processor 120 can require the first entity 103 to enter login credentials to access the profiles being identified in the registry system 140.

The data sources 130 can include, for example, one or more sensors and/or devices such as cameras, microphones, accelerometers, radio frequency identification (RFID) tags, infrared (IR) sensors, wearable devices, mobile phones, computers, tablet devices, etc. The data sources 130 can be located within an environment of the procedure 102, such as a room, a building, and/or other types of physically locations. In an example, a camera among the data sources 130 can detect physical locations and movements of entities including personnel, patients, and mobile assets in an environment of the procedure 102. The camera can be further configured to determine a number of entities in the environment of the procedure 102. In an example, a microphone among the data sources 130 can record sound and generate audio files indicating a progress of the ongoing procedure 102, such as conversations between the first entity 103 and other entities involved in the procedure 102. In some examples, RFID tags and/or IR sensors among the data sources 103 can provide data indicating locations of entities and resources within the environment of the procedure 102. The data being collected by the data sources 130 can be aggregated by the processor 120 into a dataset 132, and the dataset 132 can be transmitted to the data manager module 134.

The data manager module 134 can be configured to manage sources of data among the dataset 132. For example, the data manager 134 can identify a first set of data, such as analysis data 136, from the dataset 132 to be distributed to the analyzer module 150, where the analysis data 136 can be processed by the data analyzer module 150 to analyze a context of the procedure 102. For example, the data manager 134 can identify image data and audio data, collected by cameras and microphones, respectively, and send the identified image data and audio data to the analyzer module 150. In an example, the data manager module 134 can be programmed to search in the dataset 132 for data required for context analysis, such as image data and audio data collected from the environment of the procedure 102. The data manger module 134 can identify a second set of data, such as evaluation data 137, from the dataset 132 to be distributed to the score prediction module 160, where the evaluation data 137 can be processed by the score prediction module 160 to determine a plurality of scores. The data manager module 134 can be configured to perform data preprocessing and transformation operations on data among the dataset 132, such as duplicate removal, feature representation transformation, feature tuning, feature selection, and feature construction. The preprocessing and transformation operations can allow the analysis data 136 and evaluation data 137 to be in a format appropriate for analysis by the analyzer module 150 and the score prediction module 160, respectively.

The analyzer module 150 can be configured to generate context data 152 based on the profile data 142 and the analysis data 136 received from the data manager module 134. The context data 152 can indicate a setting, ongoing activities, environment, and time associated with a current context and future context of the procedure 102. For example, the context analyzer 150 can determine a desired, or optimized, performance, quality, and outcome of the procedure 102. The generated context data 152 can be vector representations describing the setting, activity, environment, and time pertaining to an ongoing care operation (e.g., procedure 102). For example, if the procedure 102 is associated with an entity experiencing a particular condition (e.g., a patient who has suffered a heart attack), the analyzer module 150 can generate the context data 152 to include vector representations of, for example, an age of the entity, a heart rate of the entity, blood pressure of the entity, clinical conditions of the entity, interventions that have been performed on the entity (by the entity 103 or other provider entities), provider entities who have interacted with the entity, etc. The analyzer module 150 can transmit the context data 152 to the score prediction module 160.

The score prediction module 160 can be configured to predict an effectiveness score, such as a predicted score 162, of the ongoing procedure 102 based on the profile data 142, the third set of data 152, and the second set of data 137. The score prediction module 160 can implement a plurality of machine learning models 163 (“models 163”), which can include one or more different machine learning models, and the processor 120 can train the models 163 using various data received from other modules, such as the profile data 142, the context data 152, and the second set of data 137. The models 163 can include a plurality of machine learning models that can be applied to determine 1) a patient self-regulation score indicating cognitive and behavioral capacity of the second entity 104 to control oneself (e.g., the ability to understand and follow instructions), 2) a complexity score indicating multiplicity of diseases or conditions being experienced by the second entity 104, 3) a fragmentation score indicating a measurement of dispersion of the services required by the second entity 104, 4) a provider effectiveness score indicating whether the knowledge, skills, and experience of the first entity 103 is adequate and/or effective for the procedure 102, and 5) a group provider effectiveness score indicating whether the knowledge, skills, and experience of a group of providers, which may or may not include the first entity 103, are adequate and/or effective for the procedure 102. Details of the models 163 will be provided in more detail below. The score prediction module 160 can aggregate the determined scores into a set of predicted scores 164, and transmit the predicted scores 164 to the effectiveness score generation module 166 and to the recommendation module 170.

The effectiveness score generation module 166 can generate a procedure effectiveness score 168 and transmit the procedure effectiveness score 168 to the recommendation module 170. The procedure effectiveness score 168 can indicate a current effectiveness of the procedure 102. The procedure effectiveness score 168 can vary over time as new data are being collected by the data sources 130. The effectiveness score module 166 can also determine a target score 169 indicating a desired effectiveness score for the procedure 102, and can transmit the target score 169 to the recommendation module 170. The recommendation module 170 can be configured to use the predicted scores 164 and the procedure effectiveness score 168 to identify a set of requirements and associated context to change the procedure effectiveness score 168 to the target score 169 (the identification will be described in more detail below). The recommendation module 170 can generate recommendation data 172 indicating the identified requirements and associated context, such as resources and actions, that may help to achieve the target score 169. In an example, the target score 169 can be a score that maximizes a probability of success of the procedure 102. The recommendation module 170 can send the recommendation data 172 to the optimization module 180. The optimization module 180 can be configured to determine a new set of resources based on the identified requirements and associated context indicated in the recommendation data 172, where the new set of resources may maximize probability of success of the procedure 102.

In some examples, a progression rate, which can be a rate in which the procedure effectiveness score 168 varies over time, can be determined and monitored by the effectiveness score generation module 166. For example, the effectiveness score generation module 166 can determine the procedure effectiveness score 168 at different times during the ongoing procedure 102, compare the procedure effectiveness scores 168 at the different times to determine a rate of change of the procedure effectiveness score 168. Further, the progression rate of the procedure effectiveness score 168 can indicate a likelihood of a progress of the procedure 102 approaching a completion point, where the likelihood can be based on historical data (e.g., stored in registry system 140) relating to the procedure 102. In an example, historical data can indicate that procedure effectiveness scores of procedures, that are of the same procedure type as the procedure 102, increases at a particular rate (e.g., linearly, exponentially, etc.) within the fifteen minutes prior to completion of the procedures. In response to the procedure effectiveness score 168 of the procedure 102 increasing at the same particular rate, the effectiveness score generation module 166 can determine that the procedure 102 may be approaching completion within fifteen minutes. The effectiveness score generation module 166 can notify the recommendation module 170 of the anticipated completion. The recommendation module 170 can generate recommendation data 172 to indicate tasks and resources that may be needed upon the completion of the procedure 102, and indicate that these tasks and resources need to be ready within fifteen minutes.

In some examples, the effectiveness score generation module 166 can determine and monitor progression rates of procedure effectiveness scores of other procedures (of the same procedure type as procedure 102) being performed by a cohort of provider entities, such as one or more entities that may be different from the entity 103. Note that the scores of other procedures can be based on provider effectiveness scores, which may be variable over time, of the cohort of provider entities. The effectiveness score generation module 166 can perform an analysis that can include cross-referencing the progression rate of the procedure 102 being performed by the entity 103 with the progression rates of procedures being performed by the cohort of entities. In an example, the analysis can include implementing algorithms such as text similarity analysis, cohort analysis, text matching algorithms, etc. The results of such analysis can indicate a relative effectiveness of the entity 103 compared to the other entities in performing the procedure 102. The results from the analysis can be used by the effectiveness score generation module 166 to determine the target score 169. For example, the procedure effectiveness score 168 of the entity 103 performing the procedure 102 can be relative lower than an aggregated (e.g., averaged, weighted average, median, and/or other types of aggregation or combination) effectiveness score of the other entities performing the same procedure type. The effectiveness score generation module 166 can set the aggregated scores as the target score 169 to indicate that the entity 103 may need to increase the procedure effectiveness score 168 to be comparable with other entities performing the same procedure type.

In an example, the system 100 can be implemented to provide real-time assessment and monitoring of an ongoing procedure (e.g., procedure 102). For example, the first entity 103 can use a first set of resources to perform the procedure 102 on the second entity 104 under a first set of context (e.g., setting, activity, environment, time, etc.). The data sources 130 can also be located in the environment of the procedure 102. The data source 130 can monitor the ongoing procedure 102 and collect various data to generate the dataset 132. The device 110 can be located in the environment of the procedure 102. The device 110 can include input interfaces (e.g., keyboard, mouse, touchscreen) and a display configured to display a user interface, such that the first entity 103 can use the device 110 to implement the system 100, view scores and recommendations generated by the system 100, and perform actions to control the system 100. For example, the first entity 103 can input a goal of the procedure 102, such as indicating a need to transfer the second entity 104 to a second location different from the environment of the procedure 102, to undergo a new procedure different from the procedure 102. The system 100 can output the current procedure effectiveness score 168 and the target score 169, and can output recommendations to the first entity 103 to recommend additional resources and context that may assist the first entity 103 to achieve the target score 169. For example, the recommendation can indicate that an additional amount of a particular medication may help to alleviate current condition of the second entity, and if the condition of the second entity is alleviated to a certain level, the procedure effectiveness score 168 may be changed towards the target score 169. The system 100 can also send a notification to another system in the second location to indicate a need to prepare a new set of resources for the second procedure upon transferring the second entity 104 to the second location.

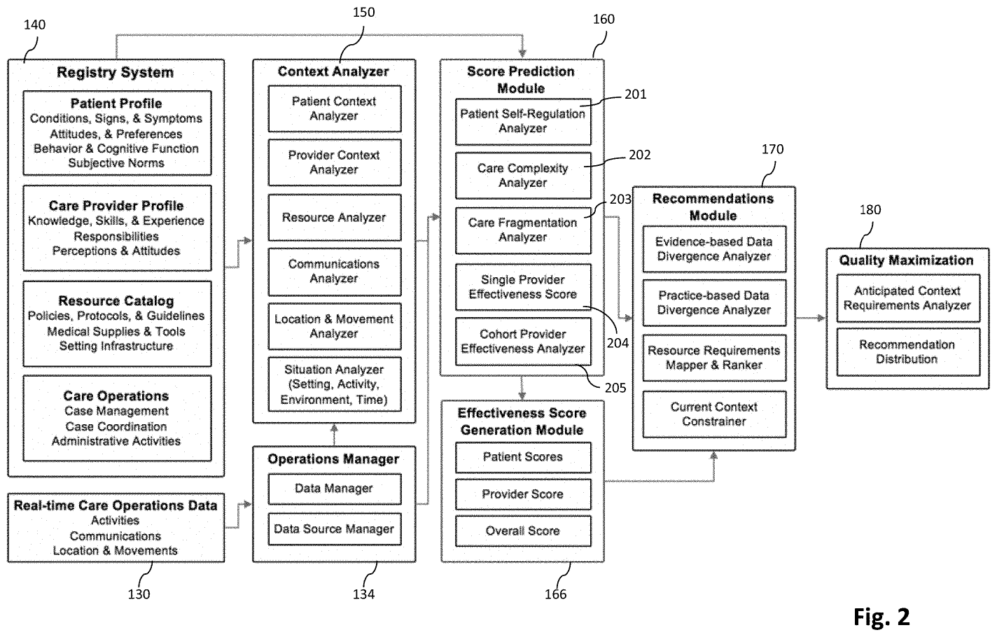

FIG. 2 is a diagram showing additional details of the example computer system of FIG. 1 that can implement operations management based on predicted operations effectiveness in one embodiment. FIG. 2 may include components that are labeled identically to components of FIG. 1, which are not described again for the purposes of clarity. The description of FIG. 2 may reference at least some of the components of FIG. 1.

The models 163 can include a first model 201 or analyzer that can be applied or run by the processor 120 to determine a patient self-regulation score, denote as S_{SRG}, indicating cognitive and behavioral capacity of the second entity 104 to control oneself (e.g., the ability to understand and follow instructions). S_{SRG} (shown below) can be based on one or more predictor variables, such as n predictor variables that can include at least a cognitive variable x_{cf}, behavior variable x_{be}, preference variable xₚᵣ, and subjective norms sₙ. Subjective norms sₙ can be, for example, a variable representing an individual's perception of whether an action shall be performed based on factors such as opinions and social pressure from other entities. The subjective norm variable can be, for example, expressed as a sum of various perceptions based on different factors associated with the entity 104 and the procedure 102 (e.g., whether the entity 104 is confident in an outcome of the procedure 102 based on different opinions). For example, the first model 201 can predict that the entity 104 may decide to undergo the procedure 102 in response to receiving an opinion from a known entity (e.g., a family member). These predictor variables can be derived, by the score prediction module 160, from the profile data 142, the context data 152, and the evaluation data 137:

S_{SRG}(W, X_{SRG})=W₀+W₁x_{cf}+W₂X_{be}+W₃xₚᵣ+W₄xₛₙ+ . . . +WₙXₙ

where the vector w=(w₀, w₁, wₙ) is a vector of weights w₀, w₁, wₙ for the n predictor variables. The predictor variables x_{SRG}=(x_{cf}, x_{be}, xₚᵣ, sₙ) may change over time, at different rates, for one or more patients or client entity. The processor 120 can be configured to continuously train the model 201 using updated and different values of these predictor variables. The training of the model 201, in some examples, can change the values of the weights among the vector w.

The models 163 can include a second model 202 or analyzer that can be applied or run by the processor 120 to determine a complexity score, denoted as S_{CCS}, indicating multiplicity of diseases or conditions being experienced by the second entity 104. S_{CCS} (shown below) can be as a function of a comorbidity index variable x_{ci}, medication regimen complexity index variable x_{mrci}, and a number of care services needed variable xₙₛₑᵣᵥ, of the second entity 104, where the variables x_{ci}, x_{mrci}, and xₙₛₑᵣᵥ can be obtained from the profile data 142:

S_{CCS}(u, x_{CCS})=u₀+u₁x_{ci}+u₂x_{mrci}+u₃xₙₛₑᵣᵥ

where the vector u=(u₀, u₁, u₂, u₃) is a vector of weights u₀, u₁, u₂, u₃. The predictor variables x_{CCS}=x_{ci}, x_{mrci}, xₙₛₑᵣᵥ may change over time, at different rates, for one or more patients or client entity. The processor 120 can be configured to continuously train the model 202 using updated and different values of the variables x_{ci}, x_{mrci}, and xₙₛₑᵣᵥ. The training of the machine learning model 202, in some examples, can change the values of the weights among the vector u.

The models 163 can include a third model 203 or analyzer that can be applied or run by the processor 120 to determine a fragmentation score, denoted as S_{CF}, indicating a measurement of dispersion of the services required by the second entity 104. S_{CF} (shown below) can be a function of the total number of clinic visits needed n, the total number of clinics to visit k, and the total number of visits to a specific clinic needed nk, and S_{CF} can be determined by:

$${S}_{CF} = \frac{1}{M} {\sum }_{i = 1}^{m} \frac{{n}^{2} - {Σ}_{1}^{k} {nk}^{2}}{n ( n - 1 )}$$

where m denotes an upper limit of summation for M entities (client entities such as patients) belonging to a cohort of client entities with similar characteristics. The processor 120 can be configured to train the model 203 using different values of n, k, m, and M.

The models 163 can include a fourth model 204 or analyzer that can be applied or run by the processor 120 to determine a provider score, denoted as S_{P}, indicating whether the knowledge, skills, and experience of the first entity 103 is adequate and/or effective for the procedure 102. In other words, the provider score S_{P} can be a provider effectiveness score of the provider entity P, with respect to the procedure being performed by the provider entity P. Sp can be a function of h variables, which may include provider education variable x_{ed}, skills variable xₛₖ, work experience variable x_{we}, responsibilities variable xᵣₑₛₚ, and j actions variables x_{act}_{₁}, . . . , x_{act}_{ⱼ}, in the procedure 102:

S_{P}(v, xₚ)=v₀+v₁x_{ed}+v₂xₛₖ+v₃x_{we}+v₄xᵣₑₛₚ+v₅x_{act}_{₁}+ . . . +vₕx_{act}_{ⱼ}

where the vector v=(v₀, v₁, vₕ) is a vector of weights v₀, v₁, vₕ. The action variables x_{act}_{₁}, . . . , x_{act}_{ⱼ} can represent different actions that may be taken by the entity 103 performing the procedure 102, such as performing a certain operation, asking for assistance from other provider entities, requesting certain resources and equipments, and/or other actions. In some examples, the weights (v₅, . . . , vₕ) can indicate a likelihood of the entity 103 performing an action represented by a corresponding action variable x_{act}_{₁}, . . . , x_{act}_{ⱼ}. The variables being used to determine S_{P} may change over time, at different rates, for one or more provider entities. The processor 120 can be configured to continuously train the model 204 using updated and different values of these variables, and the training of the model 204 can change the values of the weights among the vector v.

The models 163 can include a fifth model 205 or analyzer that can be applied or run by the processor 120 to determine a group provider or a cohort score, denoted as S_{C}, indicating whether the knowledge, skills, and experience of a group of providers, which may include the first entity 103, are adequate and/or effective for the procedures being performed by the group of providers. In an example, the entity 103 may be assigned to perform the procedure 102 on the entity 104, and another group of z provider entities may be assigned to perform the procedure 102 on other client entities. The system 100 can determine individual provider score for each one of the z provider entities S_{P1}, S_{P2}, . . . , S_{Pz}, and determine a weighted average S_{C}(y, S_{P}) of these provider scores:

S_{C}(y, S_{P})=y₁S_{P1}+y₂S_{P2}+ . . . +Y_{z}S_{Pz}

where the vector y=(y₀, y, . . . ,yₕ) is a vector of weights y₀, y₁, . . . , y_{z}. The provider scores S_{P} may change over time, at different rates, for one or more provider entities. The processor 120 can be configured to continuously train the model 205 using updated and different values of S_{P}, and the training of the model 205 can change the values of the weights among the vector y.

The effectiveness score module 166 can determine the procedure effectiveness score 168, denoted as S_{CPES}, as a function (a weighted average) of the predicted scores 162 (S_{SRG}, S_{CCS}, S_{CF}, S_{P}, S_{c}):

S_{CPES}=f(S_{SRG}, S_{CCS}, S_{CF}, S_{P}, S_{c})=c₁S_{SRG}+c₂S_{CCS}+c₃S_{CF}+c₄S_{P}+c₅S_{C}

where the vector c=(c₀, c, . . . , c₅) is a vector of weights c₀, c₁, . . . , c₅ for the scores 162.

In an example, the first entity 103 can perform the requirements indicated by the recommendation data 172 to continue the procedure 102. The data sources 130 can collect new data that indicate a new set of contexts and profiles associated with the first entity 103, the second entity 104, and the procedure 102. For example, the recommendation data 172 can indicate an additional amount of medication to be administered to the second entity 104. The first entity 103 may administer the additional amount of medication, and the second entity's condition may improve in response to the added medication. The second entity's positive response to the added medication can be collected by the data sources 130 and logged in the registry system 140, to update the second entity's profile indicating that the second entity response positively to this dosage of medication. The data sources 130 can also collect data, such as images and audio, indicating that the first entity has performed the requirements indicated by the recommendation data 172. The collected data can be analyzed by the analyzer module 150 and the results from the analysis can be logged into the registry system 140 to update the first entity's profile indicating the first entity typically inclines to follow the recommendations outputted by the system 100. Further, a profile of the procedure 102 can also be updated to indicate that this medication may provide a positive context (e.g., second entity responded positively) for the same type of procedures. Thus, the new data being collected in response to performing the requirements indicated by the recommendation data 172 can be used to update the registry system 140, where the updated data in the registry system 140 can be used by the score prediction module 160 to continuously train the models 201, 202, 203, 204, 205.

The recommendation module 170 can identify requirements and associated context required to achieve the target score 169 based on the predicted scores 162 and the procedure effectiveness score 168. The recommendation module 170 can analyze deviations between the procedure effectiveness score 168 and the target score 169, where these deviations can indicate 1) a current coordination plan of the procedure 102 and recommended coordination plans of the procedure 102, and 2) current patient coordination plan and common or frequent care coordination plans derived from retrospective pattern-mining of electronic health records (e.g., among the registry system 140 and/or data sources 130) of client entities of the same cohort (e.g., population similar to the second entity 104). The recommendation module 170 can be further configured to train or learn machine learning models (or rules) that map different values of the procedure effectiveness score 168 to different values of the determined deviations. The recommendation module 170 can identify specific recommendations and requirements based on these mappings. For example, a procedure effectiveness score E₁ being mapped to a deviation D₁ may be associated with a first set of requirements (e.g., actions, resources, equipment, associated with the procedure 102), and the procedure effectiveness score E₁ being mapped to another deviation D₂ may be associated with a second set of requirements.

The recommendation module 170 can determine a protocol divergence score Divₚᵣₒₜ, as a Jaccard distance between the current coordination plan plan_{cur}, associated with the procedure effectiveness score 168, and a protocol-recommended coordination plan planₚᵣₒₜ associated with the target score 169, such as:

$${Div}_{prot} = 1 - \frac{\vert {plan}_{cur} ⋂ {plan}_{prot} \vert}{\vert {plan}_{cur} ⋃ {plan}_{prot} \vert}$$

where plans can be sets of codes and/or discretized action items for a given care coordination instance. For example, the set plan_{cur} can indicate a set of actions to respond to particular instances or events associated with the procedure 102, and items defined by the set plan_{cur} can be identified from care coordination plan documents generated by care providers (e.g., provider entities such as entity 103) in an ongoing care operation (e.g., procedure 102). The set planₚᵣₒₜ can be identified from one or more existing care protocols and can describe recommended actions for a given care coordination instance of the procedure 102. For example, the set planₚᵣₒₜ can indicate actions, under a particular protocol, that are recommended to respond to particular instances or events similar to the instances and events relating to the procedure 102. In some examples, the procedure effectiveness score 168 can be a score achieved by performing the items indicated by the set plan_{cur}, and the target score 169 can be achievable by performing the items indicated by the set planₚᵣₒₜ.

The recommendation module 170 can determine a practice divergence Div_{prac} as a Jaccard distance between the current care plan plan_{cur} and an expected care plan planₑₓₚ based on historical data stored in the registry system 140 associated with client entities that are similar to the second entity 104:

$${Div}_{prac} = 1 - \frac{\vert {plan}_{cur} ⋂ {plan}_{exp} \vert}{\vert {plan}_{cur} ⋃ {plan}_{exp} \vert}$$

The recommendation module 170 can determine a ranked list of recommendations L_{rec} and requirements L_{req}, as a function of the procedure effectiveness score 168 (S_{CPES}), the protocol divergence Divₚᵣₒₜ, the practice divergence Div_{prac}, and the list of available resources L_{req}, such as a rule-based mapping function that maps care progression effectiveness scores (or procedure effectiveness scores) and care coordination plans deviation scores to specific recommendations and requirements, as follows:

L_{rec}, L_{req}=f(S_{CPES}, Divₚᵣₒₜ, Div_{prac}, Lᵣₑₛ)

The recommendation module 170 can be further configured to predict a highest achievable effectiveness score based on the recommendation data 172. The highest achievable effectiveness score S_{CPES_high}, can be a function of the procedure effectiveness score 168 (S_{CPES}), and the suggested recommendations L_{rec}, such as a regression function that takes as input the effectiveness score, as well as the list of recommendations (that may be transformed into dummy variables) and generates a new highest effectiveness score based on the inputs:

S_{CPES_high}=f(S_{CPES}, L_{rec})=w₁S_{CPES}+w₂L_{rec}

The optimization module 180 can be configured to determine a new set of resources based on the identified requirements and associated context indicated in the recommendation data 172. In an example, based on the procedure effectiveness score S_{CPES}, the suggested recommendations L_{rec}, and the highest achievable care effectiveness score S_{CPES_high}, the optimization module 180 can apply a context constraining function that may determine the new set of resources (e.g. medications, tools, supplies, etc.) based on preferences, expert provider recommendations, and other contextual factors that may be indicated by the profile data 142.

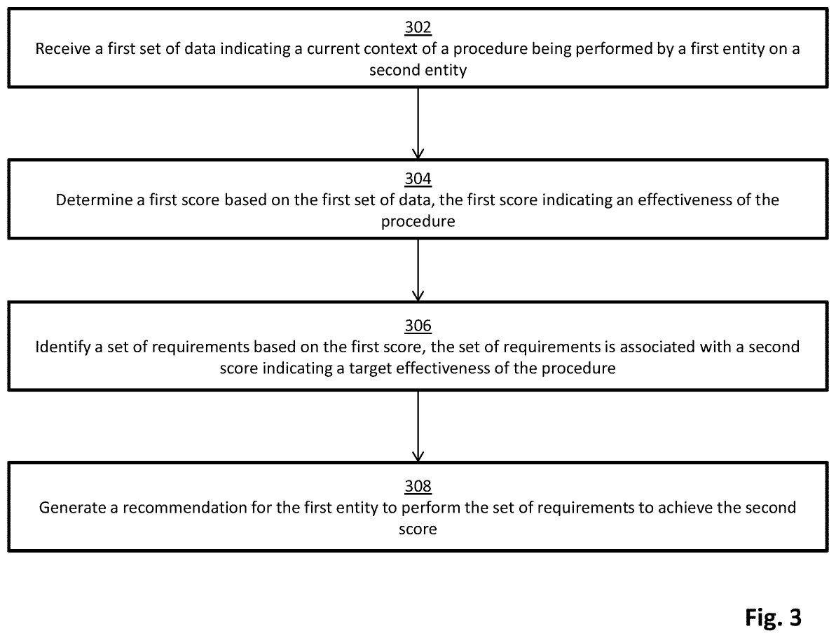

FIG. 3 is a flow diagram illustrating a process that can be performed by a processor to implement operations management based on predicted operations effectiveness in one embodiment. An example process may include one or more operations, actions, or functions as illustrated by one or more of blocks 302, 304, 306, and/or 308. Although illustrated as discrete blocks, various blocks may be divided into additional blocks, combined into fewer blocks, eliminated, or performed in parallel, depending on the desired implementation. The description of the flow diagram of FIG. 3 may reference at least some of the components of FIGS. 1-2.

The process can begin at block 302. At block 302, a processor can receive a first set of data indicating a current context of a procedure being performed by a first entity on a second entity. In some examples, the first set of data can include one or more of a profile of the first entity, a profile of the second entity, a profile of the procedure, a profile of a set of resources associated with the procedure, and a plurality of sensor data collected from a plurality of sensors.

The process can continue from block 302 to block 304. At block 304, the processor can determine a first score based on the first set of data. The first score can indicate an effectiveness of the procedure. In some examples, the determination of the first score can include 1) running a first machine learning model with the first set of data to determine a first intermediary score indicating a self-regulation of the second entity, 2) running a second machine learning model with the first set of data to determine a second intermediary score indicating a complexity of a condition of the second entity, 3) running a third machine learning model with the first set of data to determine a third intermediary score indicating a measurement of dispersion of the services required by the second entity, 4) running a fourth machine learning model with the first set of data to determine a fourth intermediary score indicating an effectiveness of the first entity on the procedure, and 5) running a fifth machine learning model with the first set of data to determine a fifth intermediary score indicating a weighted average of individual effectiveness of a plurality of provider entities.

The process can continue from block 304 to block 306. At block 306, the processor can identify a set of requirements based on the first score. The set of requirements can be associated with a second score indicating a target effectiveness of the procedure. In some examples, the identification of the set of requirements can include determining a deviation between the first score and the second score.

The process can continue from block 306 to block 308. At block 308, the processor can generate a recommendation for the first entity to perform the set of requirements to achieve the second score. In some examples, the processor can obtain a second set of data associated with the procedure and the recommendation, and can train the first, second, third, fourth, and fifth machine learning models using the second set of data.

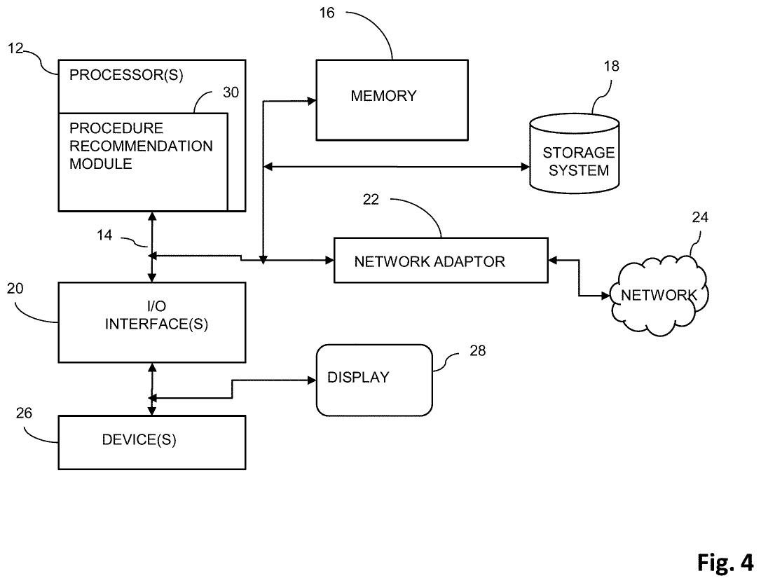

FIG. 4 illustrates a schematic of an example computer or processing system that may implement operations management based on predicted operations effectiveness in one embodiment of the present disclosure. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of well-known computing systems, environments, and/or configurations that may be suitable for use with the processing system shown in FIG. 4 may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, supercomputers, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network.

In a distributed cloud computing environment, program modules may be located in both local and remote computer system storage media including memory storage devices.

The components of computer system may include, but are not limited to, one or more processors or processing units 12, a system memory 16, and a bus 14 that couples various system components including system memory 16 to processor 12. The processor 12 may include a module 30 (e.g., procedure recommendation module 30) that performs the methods described herein. The module 30 may be programmed into the integrated circuits of the processor 12, or loaded from memory 16, storage device 18, or network 24 or combinations thereof.

Bus 14 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system may include a variety of computer system readable media. Such media may be any available media that is accessible by computer system, and it may include both volatile and non-volatile media, removable and non-removable media.

System memory 16 can include computer system readable media in the form of volatile memory, such as random access memory (RAM) and/or cache memory or others. Computer system may further include other removable/non-removable, volatile/non-volatile computer system storage media. By way of example only, storage system 18 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 14 by one or more data media interfaces.

Computer system may also communicate with one or more external devices 26 such as a keyboard, a pointing device, a display 28, etc.; one or more devices that enable a user to interact with computer system; and/or any devices (e.g., network card, modem, etc.) that enable computer system to communicate with one or more other computing devices. Such communication can occur via Input/Output (I/O) interfaces 20.

Still yet, computer system can communicate with one or more networks 24 such as a local area network (LAN), a general wide area network (WAN), and/or a public network (e.g., the Internet) via network adapter 22. As depicted, network adapter 22 communicates with the other components of computer system via bus 14. It should be understood that although not shown, other hardware and/or software components could be used in conjunction with computer system. Examples include, but are not limited to: microcode, device drivers, redundant processing units, external disk drive arrays, RAID systems, tape drives, and data archival storage systems, etc.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

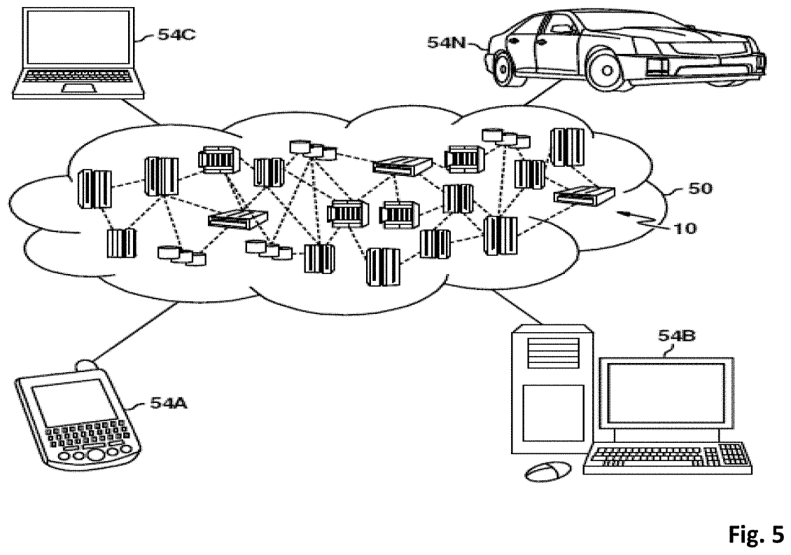

FIG. 5 depicts a cloud computing environment according to an embodiment of the present invention. It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

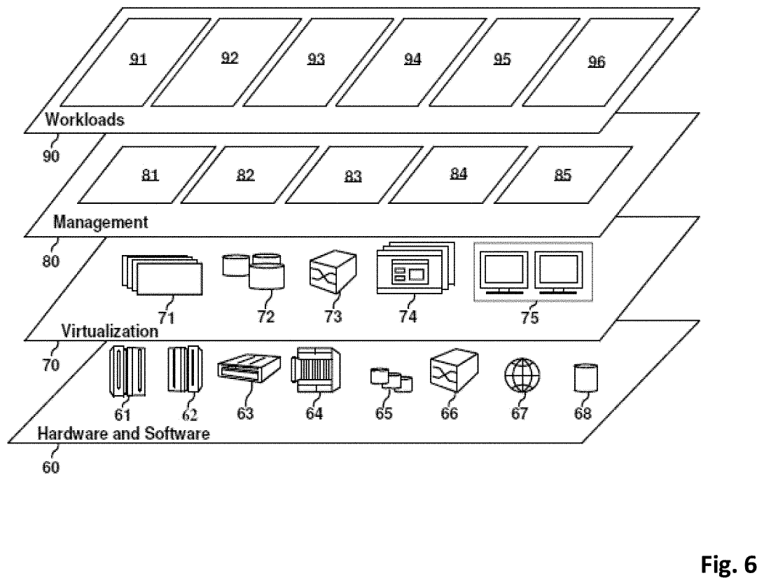

FIG. 6 depicts abstraction model layers according to an embodiment of the present invention. Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and procedure recommendation generation 96.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

We claim:

1. A computer-implemented method comprising:
receiving, by a processor, a first set of data indicating a current context of a procedure being performed by a first entity on a second entity;
determining, by the processor, a first score based on the first set of data, wherein the first score indicates an effectiveness of the procedure;
identifying, by the processor, a set of requirements based on the first score, wherein the set of requirements is associated with a second score indicating a target effectiveness of the procedure; and
generating, by the processor, a recommendation for the first entity to perform the set of requirements to achieve the second score.

2. The computer-implemented method of claim 1, wherein the first set of data indicates a first set of resources, and the method further comprising identifying, by the processor, a second set of resources for the procedure based on the recommendation.

3. The computer-implemented method of claim 1, wherein the first set of data comprises at least one of:
a profile of the first entity;
a profile of the second entity;
a profile of the procedure;
a profile of a set of resources associated with the procedure; and
a plurality of sensor data collected from a plurality of sensors.

4. The computer-implemented method of claim 1, wherein determining the first score comprises:
running, by the processor, a first machine learning model with the first set of data to determine a first intermediary score indicating a self-regulation of the second entity;
running, by the processor, a second machine learning model with the first set of data to determine a second intermediary score indicating a complexity of a condition of the second entity;
running, by the processor, a third machine learning model with the first set of data to determine a third intermediary score indicating a measurement of dispersion of the services required by the second entity;
running, by the processor, a fourth machine learning model with the first set of data to determine a fourth intermediary score indicating an effectiveness of the first entity on the procedure;
running, by the processor, a fifth machine learning model with the first set of data to determine a fifth intermediary score indicating a weighted average of individual effectiveness of a plurality of provider entities.

5. The computer-implemented method of claim 4, further comprising:
obtaining, by the processor, a second set of data associated with the procedure and the recommendation; and
training, by the processor, the first, second, third, fourth, and fifth machine learning models using the second set of data.

6. The computer-implemented method of claim 4, further comprising:
determining, by the processor, a first rate of change of the fourth intermediary score;
determining, by the processor, a second rate of change of the fifth intermediary score; and
determining, by the processor, the second score based on the first rate of change and the second rate of change.

7. The computer-implemented method of claim 1, wherein identifying the set of requirements comprises determining a deviation between the first score and the second score.

8. A system comprising:
a memory configured to store a first set of data indicating a current context of a procedure being performed by a first entity on a second entity;
a processor comprising hardware, the processor is configured to be in communication with the memory, and the processor being configured to:
determine a first score based on the first set of data, wherein the first score indicates an effectiveness of the procedure;
identify a set of requirements based on the first score, wherein the set of requirements is associated with a second score indicating a target effectiveness of the procedure; and
generate a recommendation for the first entity to perform the set of requirements to achieve the second score.

9. The system of claim 8, wherein the first set of data indicates a first set of resources, and the method further comprising identifying, by the processor, a second set of resources for the procedure based on the recommendation.

10. The system of claim 8, wherein the first set of data comprises at least one of:
a profile of the first entity;
a profile of the second entity;
a profile of the procedure;
a profile of a set of resources associated with the procedure; and
a plurality of sensor data collected from a plurality of sensors.

11. The system of claim 8, wherein to determine the first score, the processor is configured to:
run a first machine learning model with the first set of data to determine a first intermediary score indicating a self-regulation of the second entity;
run a second machine learning model with the first set of data to determine a second intermediary score indicating a complexity of a condition of the second entity;
run a third machine learning model with the first set of data to determine a third intermediary score indicating a measurement of dispersion of the services required by the second entity;
run a fourth machine learning model with the first set of data to determine a fourth intermediary score indicating attributes of the first entity;
run a fifth machine learning model with the first set of data to determine a fifth intermediary score indicating attributes of a plurality of entities associated with the first entity.

12. The system of claim 11, wherein the processor is further configured to:
obtain a second set of data associated with the procedure and the recommendation; and
train the first, second, third, fourth, and fifth machine learning models using the second set of data.

13. The system of claim 11, wherein the processor is further configured to:
determine a first rate of change of the fourth intermediary score;
determine a second rate of change of the fifth intermediary score; and
determine the second score based on the first rate of change and the second rate of change.

14. The system of claim 8, wherein to identify the set of requirements, the processor is configured to determine a deviation between the first score and the second score.

15. A computer program product for monitoring an ongoing procedure, the computer program product comprising a computer readable storage medium having program instructions embodied therewith, the program instructions executable by a processor of a device to cause the device to:
receive a first set of data indicating a current context of a procedure being performed by a first entity on a second entity;
determine a first score based on the first set of data, wherein the first score indicates an effectiveness of the procedure;
identify a set of requirements based on the first score, wherein the set of requirements is associated with a second score indicating a target effectiveness of the procedure; and
generate a recommendation for the first entity to perform the set of requirements to achieve the second score.

16. The computer program product of claim 15, wherein the first set of data indicates a first set of resources, and the method further comprising identifying, by the processor, a second set of resources for the procedure based on the recommendation.

17. The computer program product of claim 15, wherein to determine the first score, the program instructions are further executable by the processor of the device to cause the device to:
run a first machine learning model with the first set of data to determine a first intermediary score indicating a self-regulation of the second entity;
run a second machine learning model with the first set of data to determine a second intermediary score indicating a complexity of a condition of the second entity;
run a third machine learning model with the first set of data to determine a third intermediary score indicating a measurement of dispersion of the services required by the second entity;
run a fourth machine learning model with the first set of data to determine a fourth intermediary score indicating an effectiveness of the first entity on the procedure;
run a fifth machine learning model with the first set of data to determine a fifth intermediary score indicating a weighted average of individual effectiveness of a plurality of provider entities.

18. The computer program product of claim 17, wherein the program instructions are further executable by the processor of the device to cause the device to:
obtain a second set of data associated with the procedure and the recommendation; and
train the first, second, third, fourth, and fifth machine learning models using the second set of data.

19. The computer program product of claim 17, wherein the program instructions are further executable by the processor of the device to cause the device to:
determine a first rate of change of the fourth intermediary score;
determine a second rate of change of the fifth intermediary score; and
determine the second score based on the first rate of change and the second rate of change.

20. The computer program product of claim 15, wherein to identify the set of requirements, the program instructions are further executable by the processor of the device to cause the device to determine a deviation between the first score and the second score.
